(19) United States
(12) Patent Application Publication
(10) Pub. No.: US 2005/0251885 A1
(43) Pub. Date: Nov. 10, 2005

(54) Method for enhancing yield of recombinant protein production from plants

(75) Inventors: Dominique Michaud, Quebec (CA); Daniel Rivard, Quebec (CA); Raphael Anguenot, Quebec (CA); Sonia Trepanier, Quebec (CA); Louis-Philippe Vezina, Quebec (CA); France Brunelle, Quebec (CA)

Correspondence Address: OGILVY RENAULT LLP, 1981 MCGILL COLLEGE AVENUE, SUITE 1600, MONTREAL, QC, H3A2Y3, CA

(73) Assignee: UNIVERSITE LAVAL, QUEBEC (CA)

(21) Appl. No.: 10/519,843

(22) PCT Filed: Jul. 28, 2003
(86) PCT No.: PCT/CA03/01141

§ 371 Date: Jul. 4, 2005

Related U.S. Application Data
(60) Provisional application No. 60/398,779, filed on Jul. 29, 2002.

(51) Int. Cl. C12N 9/99; A01H 1/00; C12N 15/82

(52) U.S. Cl. 800/288; 435/184

(57) ABSTRACT

The present invention relates to a method for enhancing the yield of recombinant protein produced in genetically transformed plants. The invention most particularly relates to a method for preventing the undesirable proteolysis of recombinant proteins after harvest of the plant, during processing of the products from the plants. Especially, this invention focuses on introducing protease inhibitors in plants to prevent undesirable proteolysis of recombinant proteins at the time of cell disruption during the extraction process.

### TECHNICAL FIELD

The present invention relates to a method for enhancing the yield of recombinant protein produced in genetically transformed plants. The invention most particularly relates to a method for preventing the undesirable proteolysis of recombinant proteins after harvest of the plant, during processing of the products from the plants. Especially, this invention focuses on introducing protease inhibitors in plants to prevent undesirable proteolysis of recombinant proteins at the time of cell disruption during the extraction process.

### BACKGROUND ART

Recombinant expression of proteins is widely used to produce proteins of interest. Commonly used host systems are bacteria, yeast, insect cells, mammalian cells, animals and plants. However, recombinant protein expression is often impaired due to a multitude of factors. In particular, the yield of recombinant protein production is closely associated with the stability of the protein during the accumulation and the extraction processes.

In plants, several recombinant proteins have been produced with success but the primary problem encountered is the low level of recombinant protein recovery. One cause of low yield is the activity of proteases that degrade proteins.

In plants, interactions between recombinant proteins and proteases are not well defined at this time, but it is known that plants possess several non-specific proteases in their vacuoles. Leaf vacuolar proteases that are active in the mildly acidic pH range, may significantly alter the stability and integrity of recombinant proteins, and then decrease the yield of production of intact proteins.

Plant proteases may degrade recombinant proteins during two critical steps of the process of protein production. The degradation may occur, 1) in planta, during accumulation of the protein, and 2) ex planta, at the time of cell disruption during the extraction process. The latter may be of greater importance, since in this step, cell disruption liberates a pool of proteases from all parts and cell compartments of the plant. For example, it has been reported that the rice cystatin I (OC-1), a clinically useful protein, is accumulated in a stable form in the cytoplasm of transgenic potato leaf cells, but is degraded by proteases at the time of extraction (Michaud and Yelle, 2000, Michaud Ed., Austin Tex., pp. 195-206).

The basic process for extracting recombinant proteins from plant leaves generally begins with disintegrating a plant biomass and pressing the resulting pulp to produce a green juice. The green juice typically contains various proteins including proteases and a green pigmented material. It is of no use to achieve a high accumulation of recombinant protein in planta if the level ex planta, during the extraction process is decreased drastically by the activity of proteases. This invention focuses on the prevention of proteolysis occurring ex planta at the time of cell disruption during the extraction process.

Various methods in the art are suggested to protect recombinant protein against degradation by proteases.

So far, research has mainly focused on decreasing proteolytic degradation in planta. For example, one strategy to overcome the proteolysis problem in plants is to target proteins to alternative organelles and direct their accumulation in sub-cellular compartments where the protein is more stable. Different studies have demonstrated an increase in intracellular accumulation of a protein of interest, such as antibodies or vicilin when targeted to the endoplasmic reticulum using the carboxy-terminal signal KDEL (Tabe et al., 1995, J. Plant Sci. 73:2752-2759), instead of being targeted to the vacuole. However, although this strategy helps prevent proteolysis during expression of recombinant proteins, it does not reduce the risk of proteolysis at the time of extraction. During extraction, plant cells are disrupted and then release various compounds into the medium, including proteases, that may severely alter the integrity of recombinant proteins.

Another strategy is the alteration of proteolytic metabolism in planta. In one example, plants are genetically modified to decrease or eliminate the activity of specific proteases such as vacuolar processing enzymes (VPE's) as disclosed in U.S. Patent application No. 2002/0108149. In another example, catabolic processes including proteolysis are suppressed by delaying organ senescence (Int. Patent Publication No. WO01/61023). Again, these strategies may prevent degradation of recombinant proteins during their accumulation in planta, but do not reduce the risk of proteolysis during the extraction process. Additionally, these strategies are limited to alteration of proteolytic metabolism or/and proteases that are non-essential for plant development.

Classical methods to reduce the degradation of recombinant proteins ex planta during extraction, consist in quickly adjusting the pH of the extraction buffer (e.g. to pH 7) and/or in including low-molecular-weight protease inhibitors in the extraction buffer.

It is known in the art that an acidic pH increases the degradation of protein in the extraction mixture. The pH adjustment method is a viable method to limit the degradation of recombinant protein in the extraction mixture. However, this method is not effective with all recombinant proteins. Additionally, the use of an acidic pH to precipitate proteins in the extraction mixture and to isolate the soluble fraction containing the recombinant protein of interest, is a very useful method to partially purify these proteins, and thus maintaining pH to 7 is a constraint one would wish to eliminate at industrial scale. In the case of recombinant protein production in plants, this possibility is of great interest since the vast diversity of proteins from plants and the stringent purity requirement in industrial and medical applications requires an efficient and economical procedure for their isolation and purification.

The other classical method to prevent proteolysis, which consists in the addition of low-molecular-weight protease inhibitors, such as phenylmethyl sulfonyl fluoride (PMSF) or chymostatin in the extraction mixture, could be useful in a small-scale production.

However, this method is not economically suitable on an industrial scale of plant recombinant protein production, where proteins need to be produced cost-effectively in large amounts.

Considering the costly process of producing recombinant proteins in plants, it is desirable to obtain high production levels of recombinant proteins. Especially, since recombinant protein levels at the time of cell disruption during the extraction process should be high, and it is of no use to achieve a high level of recombinant protein in planta if the resulting level after extraction is comparatively low. In this context, new cost-effective methods are needed to reduce degradation of recombinant proteins by plant proteases released in the medium after cell/tissue disruption.

### DISCLOSURE OF THE INVENTION

One aim of the present invention is to provide a method for increasing the recovery yield of a recombinant protein in plant cells without significantly altering the natural physiology of the plant cells, comprising neutralizing the activity or the action of at least one plant protease involved in the degradation of the recombinant protein with an inhibitor released from the plant cell at the time said plant cells are disrupted. The plant cells are from a plant or from an in vitro culture. It will be recognized by those skilled in the art that the neutralizing is partial or total, and can occur when processing the plant cells for extracting the recombinant protein, and that plant cells are disrupted when performing a process for extracting the recombinant protein. The inhibitor is preferentially recombinantly produced in the plant cells transformed with an expression cassette comprising a promoter operably linked thereto. Also, the inhibitor can be linked to a leader peptide, a signal peptide or an anchorage peptide or a protein to lead or anchor said inhibitor to a cell part or extracellular compartment in a manner to protect the recombinant protein from the activity of a plant protease during the extraction process. For example, but not limited to, the cell part can be an organelle selected from the group consisting of a mitochondria, a chloroplast, a storage vacuole, the endoplasmic reticulum, and the cytosol. Also, the inhibitor can be encoded by a gene under control of a constitutive or an inducible promoter or a tissue or development specific promoter.

Targeted proteases to be inhibited or neutralized can be selected from the group consisting of a cysteine protease, an aspartate protease, a metallo protease, a serine protease, a threonine protease, and a multispecific protease.

In accordance with one particular aspect of the present invention, the inhibitor significantly does not interfere with the activity of the protease to preserve the physiology or the growth of the plant cells or plant containing the plant cells.

Another aspect of the invention is to provide a method for neutralizing, or modulating in planta an inhibitor is selected from the group consisting of an antibody or a fragment thereof, a sens-mRNA or anti-sens mRNA, an inhibitor of transcription or a regulator thereof, an inhibitor of translation or a regulator thereof, an inhibitor of leading or signal peptide, an inhibitor of metabolic acquisition of activity of a protease, a protease-specific protease, and an affinity peptide protease leading to segregation to said protease into an organelle or a cell compartment.

Preferentially, the genetically altered plant is an alfalfa or a potatoe.

The targeted proteases to be neutralized can be a chymostatin-sensitive serine protease or a cystatin-sensitive cysteine protease.

Preferentially, the recombinant protein or inhibitor are produced in nucleus or plastids of said plant cells.

Another aim of the present invention is to provide method for increasing the recovery yield of a recombinant, protein in a plant comprising the steps of:
a) allowing production of a recombinant protein in plant cells genetically altered for modulating at least one genetic or metabolic reaction to partially or totally neutralize action or activity of at least one protease at the time of disrupting of the plant cells; and
b) recovering the recombinant protein after disrupting of the plant cells.

The plant cells are from a plant or from in vitro culture.

The action or activity of the protease can be neutralized by inhibiting its transcription or translation into an active protease, or by an inhibitor produced by the plant cells, or linking the recombinant protein with a peptide or protein in manner to protect the recombinant protein from the action or activity of the protease.

In accordance with the present invention there is provided a plant cell or a plant genetically altered to modulate at least one genetic or metabolic reaction to partially or totally neutralize the action or activity of at least one protease for improving the recovery of a recombinant protein from the plant cell or plant at the time the plant cell or cells of the plant are disrupted.

Another object of the present invention is to provide a method of introducing protease inhibitors in plants to prevent undesirable proteolysis of recombinant proteins at the time of cell disruption occurring or performed during the extraction process.

This invention is partially based on the identification of protein inhibitors efficient in inhibiting an important fraction of potato and alfalfa proteases found in crude extracts of leaves and stems. Target protease activities in potato and alfalfa have been tested for proteolytic on proteins of interest such as human fibronectin. These plant proteases show proteolytic activity against recombinant proteins of interest and the present invention provides new strategies to alter the undesirable activity of these proteases during the extraction process.

One object of the present invention is also to provide a method to enhance the yield of production of recombinant proteins in plants by preventing proteolysis after cell disruption but without negatively altering the normal metabolism or development of the host plant.

Also one object of the present invention is to provide a method to prevent proteolysis of recombinant proteins at the time of cell disruption during the extraction process, this method allowing, for example, the use of acidic pH in the extraction mixture to precipitate proteins and isolate a soluble fraction containing the recombinant protein of interest.

Another goal of the invention is the judicious choice of the inhibitor to be expressed in the plant as well as its subcellular targeting, to insure a sufficient accumulation of the inhibitor in planta and a satisfying stability of this inhibitor at the time of harvesting, stocking and extraction, in order to reach the optimal protection effect of recombinant proteins at the time of cell disruption during the extraction process.

According to one aspect of the present invention there is provided a method for enhancing the yield of production of recombinant protein in plants or plant cells, this method comprising the step of obtaining plants or plant cells co-expressing at least (a) a recombinant protein, and (b) an inhibitor of endogenous plant proteases implicated in the degradation of said recombinant protein, whereby the control expression of the inhibitor specified at (b) enables the proteolytic degradation of the recombinant protein specified at (a) to be prevented or reduced thereby increasing the recovery yield of the recombinant protein, without altering negatively the metabolism or development of the plant or plant cells.

The inhibitor may be co-expressed in the plant with the protein of interest, or fused to the protein of interest. The inhibitor may be co-expressed with the recombinant protein in the same sub-cellular compartment, or in a different one.

The use of antibodies or a fragment thereof as a protease-specific inhibitor is also another aspect of the present invention.

According to the method of the present invention, there is provided a genetic alteration, such as DNA fragment insertion into a plant to inhibit the expression of a protease. The genetic alteration may include lockout or silencing methods. The invention also includes methods in which the inhibitory effect is constitutive or inducible, which is made possible by the use of constitutive or inducible promoters.

The present invention also provides a method in which a transgenic plant expressing a recombinant protein of interest is harvested with a transgenic plant expressing at least one protease-specific inhibitor, in order to protect the protein of interest against endogenous proteases of the plant released during the cell lysis and/or the extraction procedure.

For the purpose of the present invention, the following terms are defined below.

The term “recombinant protein” as used herein is intended to mean a protein, peptide, or polypeptide that is produced by the plants or plant cells using recombinant techniques. The recombinant protein is produced through the expression of a corresponding transgene which has been introduced in the plants or plant cells to have genetically modified plants or plant cells and expressed therein.

Proteins or factors that can be recombinantly produced may for example, but not limited to, alpha.-, beta.- and .gamma.-interferons, immunoglobulins, lymphokines, such as interleukins 1, 2 and 3, growth factors, including insulin-like growth factor, epidermal growth factor, platelet derived growth factor, transforming growth factor-.alpha., -.beta., etc., growth hormone, insulin, collagen plasminogen activator, tissue plaminogen activator, thrombin, fibrinogen, aprotinin, blood factors, such as factors I to XII, histocompatibility antigens, collagen, gelatin, enzymes such as superoxide dismutase, or other mammalian proteins, particularly human proteins.

The terms “promoter” or “promoter region” or “transcriptional regulatory sequence” as used herein mean a DNA sequence, usually found upstream (5′) to a coding sequence, that controls expression of the coding sequence by controlling production of messenger RNA (mRNA) by providing the recognition site for RNA polymerase and/or other factors necessary for initiation of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of gene sequences under their control when introduced into a host as part of a suitable recombinant vector, as demonstrated by its ability to produce mRNA.

The expressions “plant cell” or “plant part” as used herein is intended to refer to plantlets, protoplasts, calli, roots, tubers, propagules, seeds, seedlings, pollen, any other plant tissues.

The term “protease” is intended to mean an enzyme that performs directly or indirectly the degradation of polypeptides into smaller peptides, fragments or amino acids, or into a form leading to the loss of the stability or activity of a protein of interest.

### BRIEF DESCRIPTION OF THE DRAWINGS

FIGS. 1A, 1B and 1C illustrate the time-course degradation of NPTII (A), human fibronectin (B) and human haemoglobin (C) by a crude extract of proteins of alfalfa leaves. The NPTII protein (A) was obtained through stable expression and extraction from potato leaves. Commercially available fibronectin (B) and haemoglobin (C) were added to crude extract of alfalfa leaves.

FIG. 2 illustrates the proteolytic activity of alfalfa (A) and potato (B) proteases in a gelatin-embedded polyacrylamide gel;

FIG. 3 illustrates the inhibition of specific alfalfa leaf proteases in alfalfa leaf with diagnostic and plant recombinant PIs;

FIG. 4 illustrates the inhibition of specific potato leaf proteases in potato leaf with diagnostic and plant recombinant PIs;

FIGS. 5A and 5B illustrate the separation of alfalfa leaf proteases by ion exchange chromatography (A), and the stabilization of human fibronectin against a major protease fraction with chymostatin and α-1-antichymotrypsin (B);

FIGS. 6A, 6B and 6C illustrate the separation of a potato leaf cathepsin D-like activity by ion exchange chromatography (A and B), and its inhibition by the aspartate proteinase inhibitor GST-CDI (C);

FIG. 7 illustrates the decrease in cathepsin D-like activity in transgenic potato lines expressing a tomato CDI transgene;

FIG. 8 illustrates the partial stabilization of recombinant NPTII in a transgenic potato line (CD21A) expressing a tomato CDI transgene, as compared to a control plant; and

FIG. 9 illustrates variations of the strategy of recombinant protease inhibitor expression in plants to hinder protease activity after cell disruption, during the protein recovery process.

### MODES OF CARRYING OUT THE INVENTION

The present invention provides new methods for enhancing the yield of recombinant protein recovered from transgenic plants or plant cells.

Also, the present invention is directed to a method for producing plant lines genetically altered to inhibit at least one protease for preserving the integrity of a recombinant protein of interest at the time of cell disruption during the extraction process.

Also, one object of the present invention is to provide a method for preventing proteolysis of recombinant proteins at the time of cell disruption during the extraction process, this method allowing the use of acidic pH in the extraction mixture to precipitate proteins and isolate a soluble fraction containing the recombinant protein of interest.

In one embodiment of the invention a protease can be identified and targeted to be inhibited as a protease specifically involved in the degradation of a recombinant protein of interest during the extraction process.

In another preferred embodiment of the invention strategies to specifically express and target the recombinant protein and the protease inhibitor are chosen so as to significantly not to affect or preserve the metabolism or development of the transgenic plant. It will be understood here that the normal physiology of a plant or plant cell in which conditions for inhibiting the activity or action of a protease at the time of recovering, including cell lysis, the protein of interest, is preferentially not altered. For example, but not limited to, a plant in which genetic modification results in inhibition of a protease therein, will grow at the same rate than a non modified plant. Under another aspect, the protein synthesis is also not altered by the conditions in the plant or plant cell resulting in the inhibition of a protease when recovering or extracting a protein of interest.

In another embodiment of the invention, a protease inhibitor can be targeted to a subcellular compartment different from the natural localization of a targeted protease in order to preserve the vital activity of the protease during the growth of the plant, and promote protection of recombinant proteins at the time of cell disruption during the extraction process of the recombinant protein.

In accordance with the present invention, there is provided a method that will give conditions causing the inhibition, partial or total, of the action or the activity of the proteases at the time a protein of interest is recovered or extracted from a plant or a plant cell. Preferentially, the method makes use of protease inhibitors, and use of sequences to genetically engineer plants or plant cells in a manner to protect from the activity of a protease the recombinant proteins produced in these transgenic plants or plant cells. Another condition of inhibiting the activity of a protease according to the present invention is that the inhibitor binds directly the protein of interest to avoid the protease to access the cleavage site for example, of binds directly the protease in order to block its action or activity.

In another embodiment of the invention the inhibitor can be chosen from the group consisting of, but is not limited to, (i) inhibitors of cysteine proteases, (ii) inhibitors of aspartate proteases, (iii) inhibitors of metallo proteases, (iv) inhibitors of serine proteases, (v) inhibitors of threonine proteases, and (vi) inhibitors with a broad range of specificity, natural or hybrid.

Alternatively, the protease inhibition according to the invention, can be performed in changing the specificity of the protease itself or the condition that cause changes in the specificity of the protease for the protein of interest during its recovering or extraction. The specificity changing or the protease for the protein of interest will preferentially not affect its activity naturally occurring in a plant or plant cell.

Different strategies can be employed to engineer plants. For example, this can be carried out, without limiting it thereto, by 1) inserting a protease inhibitor encoding gene into the genome of a plant, Another embodiment of the present invention is to provide a method in which any gene encoding a potent protease inhibitor may be introduced into the genome of a plant to reduce proteolytic activity during the extraction process which is desirable for the high-yield production of recombinant proteins. Examples of protease inhibitors that could be introduced into plants consist of, but are not limited to, the plant cystatins OCI, OCII and TMC-8, the human serpin alpha-1-anti-chymotrypsin (AACT), and the aspartate type inhibitor CDI (Tomato cathepsin-D inhibitor). For example, human serpin alpha-1-anti-chymotrypsin (AACT) could be used to inhibit alfalfa endogenous proteases while tomato CDI could be expressed in potatoe to block the endogenous aspartae proteinase. A method for introducing a protease inhibitor in alfalfa and potato is exemplified hereinbelow.

The inhibitor can be alternatively a protease propeptide.

One way to achieve protease inhibition, is also the production, in transgenic plant, of a specific antibody or an antibody fragment directed to a protease that will hinder its normal activity. This method of inhibition is dependent on the capacity of the antibody to bind to its antigen in the plant cell. Hence, it is required that the plant produces the antibody, which can be achieved by genetically transforming the plant with the transgene or transgenes needed to produce an active immunoglobulin. The production of antibodies or fragments thereof in plants is known of those skilled in the art since different antibodies have been expressed in transgenic plants including immunoglobulins (IgG, IgA and IgM), single chain antibody fragment (ScFv), fragment antigen binding (Fab), and heavy chain variable domains.

The antibody or a fragment thereof could be targeted to a different subcellular compartment from the natural localization of the targeted protease in order to preserve the vital activity of the protease during growth of the plant, and to promote protection of the recombinant protein specifically at the time of extraction or cell lysis.

One embodiment of the present invention is to provide a method that utilizes at least one DNA fragment to inhibit the expression of an endogenous protease in a genetically altered plant producing a recombinant protein.

According to another aspect of the invention, plants or plant cells are obtained with a vector useful for plant or plant cell transformation, comprising a DNA sequence encoding the recombinant protein and a DNA sequence encoding the inhibitor.

According to one aspect of the invention, transgenic plants or plant cells are obtained by transformation of whole plant, plant cells, plant protoplasts or plant plastids with one or more useful vectors comprising at least: (a) a first DNA fragment harbouring a DNA sequence encoding a recombinant protein of interest operably linked to a first promoter, fused or not to a targeting peptide to direct the protein to a particular subcellular or extracellular compartment of the plant or plant cells; and (b) a second DNA fragment harbouring a DNA sequence encoding a protease inhibitor operably linked to a second promoter, fused or not to a targeting peptide to direct the inhibitor to a particular subcellular or extracellular compartment of the plant or plant cells.

According to another aspect of the invention, plants or plant cells are obtained by crossing a first plant comprising (a) a first DNA fragment harboring a DNA sequence encoding a recombinant protein operatively linked to a first promoter, fused or not to a first targeting peptide to direct the protein to a particular subcellular or extracellular compartment of the plant or plant cells, with a second plant containing (b) a second DNA fragment harbouring a DNA sequence encoding a protease inhibitor operably linked to a second promoter, fused or not to a second targeting peptide to direct the inhibitor to a particular subcellular or extracellular compartment of the plant or plant cells.

In one embodiment of the invention, the presence or absence of a signal peptide achieves targeting of the protease inhibitor to the same subcellular or extracellular compartment as the recombinant protein of interest. Alternatively, the presence or absence of a signal peptide enables to target the inhibitor to a subcellular or extracellular compartment that is different from the recombinant protein of interest.

In one embodiment of the invention, targeted sub-cellular or extracellular compartments of the plant are chosen from the group of, but not limited to, mitochondria, plastids, storage vacuoles, endoplasmic reticulum, cytosol, and extracellular compartment.

Also, according to another aspect of the invention, transgenic plants or plant cells are obtained by genetic transformation of a plant or plant cell with a vector suitable for plastid transformation comprising the DNA sequence encoding the recombinant protein and the DNA sequence encoding the inhibitor operably linked to a promoter operative in the plastid.

Also, in one embodiment of the invention, the protease inhibitor encoding gene may be co-inserted in the plant genome with the gene of the protein of interest, in the same sub-cellular compartment or not. The inhibitor may be fused to the recombinant protein to be produced in the plant. A plant expressing one or several protease inhibitors may be crossed with a plant expressing the recombinant protein.

In another aspect of the invention, transgenic plants or plant cells are obtained by genetic transformation with a vector comprising a DNA sequence encoding the recombinant protein fused to a DNA sequence encoding the protease inhibitor operably linked with a unique promoter, and which optionally comprises the fusion of a targeting peptide to direct the fused protein and inhibitor to a particular subcellular or extracellular compartment of the plant or plant cells.

In another embodiment of the invention, expression vectors used to perform the method according to the invention may include a promoter that can be constitutive, inducible, development specific, tissue specific, or stress specific.

Also, in order to perform the method according to the invention, the activity or expression of a protease can be directly or indirectly genetically altered.

Also, part of the invention is the use of constitutive but also inducible promoters to control the expression of the inhibitor. For example, the inhibitor could be induced, or its synthesis, at the time of harvesting only, by the addition of the inducing agent prior harvesting.

Alternatively, according to another aspect of the invention the method may involved the exogenous induction of an endogenous plant inhibitor to inhibit a specific protease inhibitor at the time of harvesting to increase the recovery yield of the recombinant protein.

In accordance with the present invention, are provided methods for producing plant lines for molecular farming. Any plant species can be used to perform any method, strategy, or approach described herein to partially or totally inhibit the action of a protease against a recombinant protein of interest.

Of particular interest, the present invention can be applied to alfalfa or potato.

### EXAMPLES

The present invention will be more readily understood by referring to the following examples, that are given to illustrate the invention rather than to limit its scope.

### Example I

### Degradation of NPTII Protein by Plant Leaf Proteases

Materials and Methods

The hypothesis that degradation of specific recombinant protein can be decreased by the expression of a exogenous protease inhibitor was tested using a simple model. The neomycin phosphotransferase (NPTII) protein which is often use as selectable marker of transgenic plants was expressed in potato without the presence of any protease inhibitor protein and the degradation of the NPTII protein was monitored. In order to mimic the situation where a protease inhibitor gene would be present and expressed on the same construct as the nptII gene, a protease inhibitor gene, the tomato cathepsin-D inhibitor CDI (Werner et al, 1993, Plant Physioly 103:1473), was introduced beside the NPTII gene but without any promoter hence prohibiting CDI gene expression.

The tomato CDI-encoding DNA sequence was isolated from the expression vector pGEX-3X/CDI (Brunelle et al. 1999, Arch. Insect Biochem Physiol. 42:88-98) by digestion with BamHI and EcoRI, and subcloned between the BamHI and EcoRI cloning sites of the commercial vector pCambia 2300 (CAMBIA, Canberra, Australia). Axenically-grown plantlets of potato (Solamum tuberosum L. cultivar Kennebec) were used as source material for genetic transformation. The plantlets were maintained on MS multiplication medium (Murashige and Skoog 1962, Physiologia Plantarum 15:473-497) supplemented with 0.8% (w/v) agar (Difco, Detroit, Mich.) and 3% (w/v) sucrose, in a tissue culture room at 22° C. under a light intensity of 60 μmol/m²/s and a 16 h/day photoperiod provided by cool fluorescent lights. Leaf discs of about 10 mm in diameter were genetically-transformed using the bacterial vector Agrobacterium tumefaciens LBA4404 as described by Wenzler et al. (1989, Plant Sci. 63:79-85), except that cefotaxime, instead of carbenicillin, was used for A. tumefaciens growth control. Regenerated shoots were transferred onto selection medium with kanamycin and cefotaxime, for root regeneration and plantlet multiplication. For acclimation, the plantlets were transferred for 14 days in a growth chamber under a 24°/21° C. day/night temperature cycle, a 12-h L:D photoperiod, a light intensity of 200 mmol/m²/s and a relative humidity of 60%, before being transferred in greenhouse under standard growth conditions. Integration of the nptII (marker) transgene in kanamycin-resistant plants was confirmed by PCR, using DNA extracted from the fourth, fifth and sixth leaves (from the apex) of ˜30-cm potato plants, according to Edwards et al. (1991, Nuc. Acids Res. 19:1349).

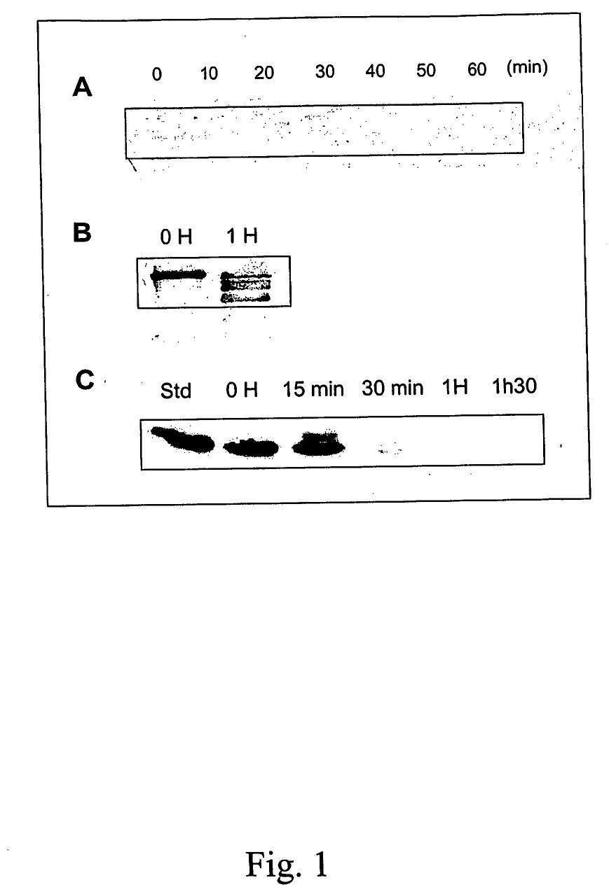

Protein extracts were prepared from PCR positive plants and subjected to a time course experiment where the degradation of the NPTII protein was monitored by Western analysis using a commercially available antibody. FIG. 1A illustrates the degradation of NPTII protein by potato leaf proteases in crude extracts from control transgenic lines expressing the nptII gene and containing the CDI gene without promoter. Detection of NPTII protein was performed by Wester blotting techniques. As seen on the Western blot (FIG. 1A), NPTII protein degradation is observed within the first 10 min. of incubation.

### Example II

### Degradation of Clinically-Useful Proteins by Plant Leaf Proteases

Materials and Methods

Other recombinant proteins may be targeted for degradation by proteolysis during the extraction procedure. In particular, this degradation may have a very negative effect for the recovery of plant made pharmaceuticals. To illustrate that this process which occurs in potato can also be found in other plants, the degradation of clinically useful proteins was monitored in leaf extracts of alfalfa. This experiment involved the addition of commercially available proteins to alfalfa leaf extract in vitro and the monitoring of the degradation of these proteins by Western analysis over a time period. In a first experiment (FIG. 1B), in vitro degradation of human fibronectin in the presence of alfalfa proteases was monitored by mixing 5 μl of alfalfa (cultivar Saranac) leaf extract prepared in 50 mM Tris-HCl pH 7.0 (1:3 w/v) containing 10 mM β-mercaptoetlianol, with 2 μg of fibronectin (Boehringer Mannheim, cat # 1080938). The mixture was incubated at 37° C. and the reaction was stopped by adding 5 μl of SDS-PAGE denaturing/loading buffer. The protein samples (T=0 and T=1 hr) were loaded on a 10% (w/v) SDS-PAGE gel, and electro-transferred onto a nitrocellulose membrane. The substrate proteins and their proteolytic fragments were immunodetected with polyclonal antibodies against human fibronectin (Sigma Aldrich, cat # F3648).

In a second experiment (FIG. 1C), in vitro stability of human haemoglobin in the presence of alfalfa proteases was monitored by mixing 20 μg of alfalfa leaf extract prepared with 20 mM Mops, pH 7,5, containing 0,1% Triton X-100, 2 mM PMSF and 10 μM chymostatin, with 200 ng of haemoglobin (Sigma, cat # H-7379), for a final volume of 8 μl. The mixture was incubated at room temperature and the reaction was stopped by adding 2 μl of denaturing/loading buffer 5× with β-mercaptoethanol at different times (0′, 15′, 30′, 1 h and 1 h30). The protein samples were loaded on a 15% (w/v) SDS-PAGE gel and electro-transferred onto a PVDF membrane. The substrate protein was immunodetected with monoclonal antibodies against human hemoglobin (Fitzgerald Cat # 10H03). The lane “Std” on the gel corresponds to 200 ng pure hemoglobin. In summary, FIG. 1 illustrates the degradation of fibronectin (B) and hemoglobin (C) in the presence of alfalfa leaf extracts, showing the hydrolytic effect of plant's endogenous proteases against these proteins. Fibronectin, for instance, is readily degraded by alfalfa (cultivar Saranac) endogenous proteases to lead intermediates finally hydrolyzed (FIG. 1B). Hemoglobin is also degraded after a 30 min. incubation with alfalfa proteases (FIG. 1C).

### Example III

### Identification of Major Protease Activities in Plant Leaf Extracts

Materials and Methods

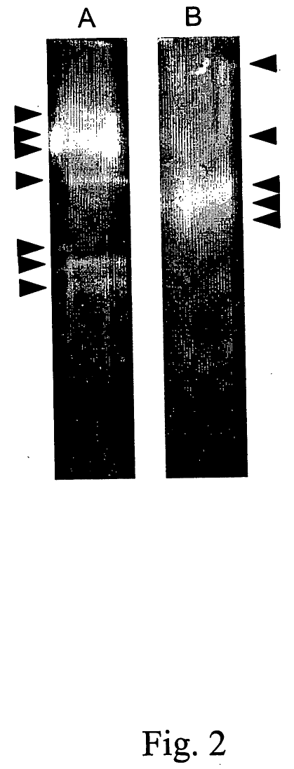

There are several kind of proteases found in different plant species. In order to characterize the major protease activities found in alfalfa and potato, crude protein extracts were obtained from the leaves of these two species. FIG. 2 illustrates the hydrolytic action of endogenous alfalfa (A) and potato (B) leaf proteases (arrows) on the degradation of gelatin. Soluble proteins were extracted (1:3 W/V) from alfalfa (cultivar Saranak) or potato (cultivar cultivarKennebec) leaves with 50 nM Tris-HCl pH 7.5, and resolved under non-reducing conditions on a 10% (w/v) SDS-polyacrylamide slab gel embedded with 0.1% (w/v) gelatin (Michaud et al., 1993, Electrophoresis 14:94-98). Proteinase renaturation was carried out by incubating the gels for 30 min at 25° C. in 2.5% (v/v) Triton X-100. Gelatinase reaction was activated by placing the gels in 100 nM citrate phosphate pH 6.0, containing 0.1% Triton X-100 and 5 mM L-cysteine, for 30 min at 37° C. Proteinases were visualized as clear (lysis) bands against a blue background, after staining with Coomassie Brilliant Blue.

This detection method would easily enable the identification of a specific protease inhibitor activity towards one of more protease activities obtained. One skilled in the art could perform similar protein extract, add the specific protease inhibitor, and detect on the gelatin gel the disappearance of lysis band which would indicate that the protease inhibitor used was able to inactivate this specific protease activity.

### Example IV

### Effect of Various Protein Inhibitors on Specific Plant Protease Activities

Materials and Methods

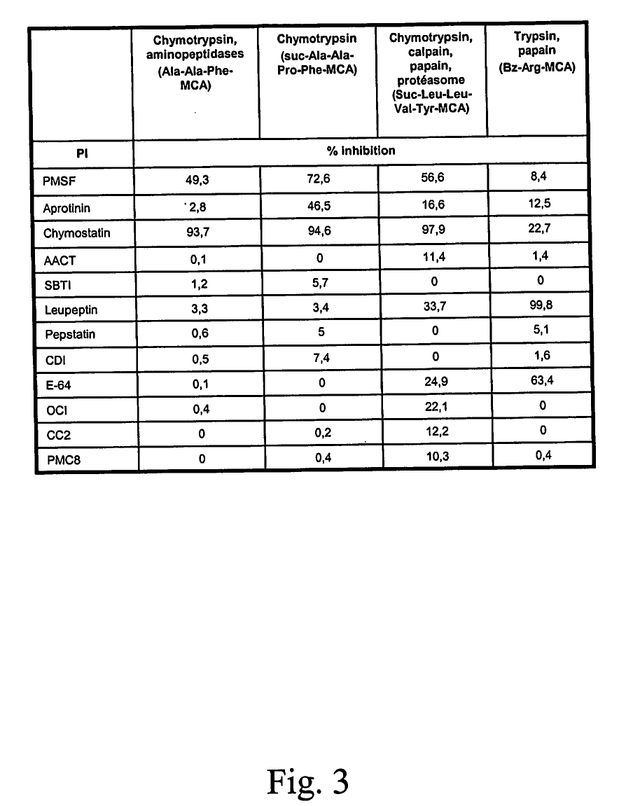

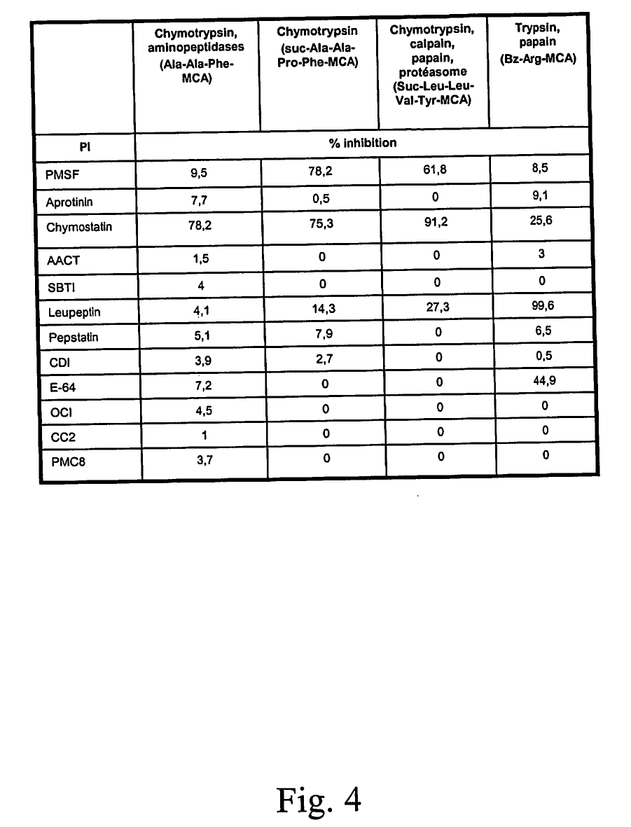

Using the method described in example III, it may be possible to identify which protease activity is responsible for the degradation of a specific clinically useful protein. From there, it would be interesting to be able to find a specific protease inhibitor to selectively abolish the protease activity. The use of synthetic fluorometric protease substrates was investigated towards this application. Fluorimetric protease substrates are useful to determined the potential of various diagnostic or recombinant PIs on the inhibition of specific plant proteases. Leaf proteins were extracted (1:3 w/v) in 50 mM Tris-HCl pH 7.5 containing 10 mM 3-mercaptoethanol, and protein content was adjusted to a final concentration of 1 mg/ml with extraction buffer. A master reaction mix was prepared by mixing 1080 μl extraction buffer, 108 μl plant extract and 12 μl of either 1 mM Ala-Ala-Phe-MCA, 1 mM suc-Ala-Ala-Pro-Phe-MCA, 1 nM suc-Leu-Val-Tyr-MCA or 1 mM Bz-Arg-MCA. One hundred μl of the master mix were dispensed in 96-well microplates and 5 μl of 100 mM PMSF (inhibitor of serine proteases), 1 mM aprotinin (inhibitor of serine proteases), 10 mM chymostatin (inhibitor of serine proteases and some cysteine proteases), 1 mg/ml α-1 antichymotrypsin (inhibitor of chyinotrypsin-like proteases), 10 mM leupeptin (inhibitor of trypsin-like proteases and some cysteine proteases), 1 mM pepstatin (inhibitor of aspartate proteases), 100 mM E-64 (inhibitor of cysteine proteases), recombinant CDI (cathepsin-D inhibitor; inhibitor of aspartate proteases), recombinant OCI (oryzacystatin I; inhibitor of cysteine proteases), recombinant CCII (corn cystatin 2; inhibitor of cysteine proteases) and recombinant PMC8 (potato multicystatin domain 8; inhibitor of cysteine proteases) were finally added to the reaction mixture. Fluorescence intensity was measured 100 times over a 5,000-sec period at 30° C. using a Fluostar Polastar Galaxy™ fluorimeter (BMG Lab Technologies), with excitation and emission filters of 485 nm and 520 mm, respectively. Protease activity, expressed in units of fluorescence per min., corresponded to the slope of the emission curve. As shown in FIGS. 3 and 4, various types of proteases may be considered as possible targets to decrease protease activities from alfalfa and potato leaves, including serine (e.g., PMSF-, aprotinin, chymotrypsin- and chymostatin-sensitive), cysteine (E-64/cystatin-sensitive) and aspartate (pepstatin-sensitive) proteases.

### Example V

### Inhibition of Fibronectin Proteolysis in Alfalfa Leaf Extracts

Materials and Methods

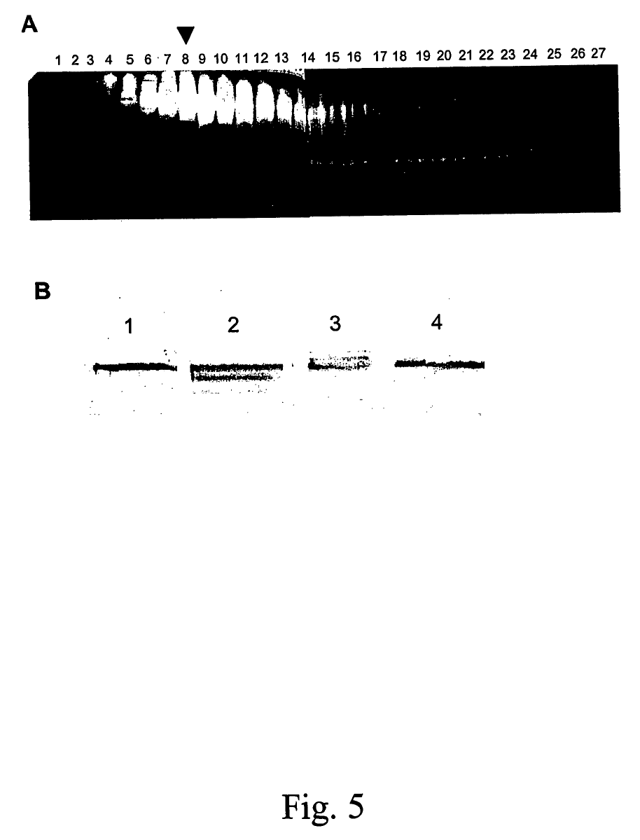

The human fibronectin was shown to be susceptible to protease degradation in alfalfa leaf extract (FIG. 1C). The following step was to demonstrate the use of various protease inhibitors to inhibit the fibronectin degradation. The stability of fibronectin was significantly increased by inhibiting alfalfa proteases with the serine-type inhibitor α-1 antichymotrypsin (FIG. 5). Firstly, a protein extract from alfalfa leaves was separated by chromatography to isolate a specific fraction containing the greatest protease activity. Alfalfa (cultivar Saranac) leaves were extracted by grinding in liquid nitrogen and resolubilization in 50 mM Tris-HCl, pH 6,8, containing 10 mM β-mercaptoethanol. The crude extract was centrifuged for 15 min at 10000 g at 4° C., and the supernatant was filtered through a 0.3 μm pore size filter. Fifteen mg of leaf proteins were then loaded on of a Mono-Q FPLC column (Pharmacia) equilibrated with extraction buffer. Proteins were eluted with a linear gradient of KCl (0 to 0.7 M) in extraction buffer, at a flow rate of 2 ml/min. Fractions of 500 μl were collected, and sample of each fraction was loaded onto a gelatin/PAGE gel (FIG. 5A). Fraction #8, which caused the highest proteolysis of gelatin in gel, was use to assess the protective effect of α-1 antichyinotrypsin.

Secondly, the identified fraction #8 was used in conjunction with various protease inhibitors to identify potential candidates for the inhibition of fibronectin proteolysis. In the experiment illustrated in FIG. 5B, 5 μl of fraction #8 was mixed with 350 ng of fibronectin and incubated at 37° C. for 15 min, in the presence of 2 μl H₂O (lane 2), 2 μl of 10 mM chymostatin (lane 3) or 2 μl α-1 antichymotrypsin (lane 4). The control (lane 1) contained 5 μl extraction of buffer instead of alfalfa proteases. The reaction was stopped after 15 min. and fibronectin was immunodetected as in FIG. 1C. As shown in FIG. 5B, the fraction eluted by Mono-Q chromatography caused significant proteolysis of fibronectin, but was prevented by inhibitors of serine-like proteases, chymostatin and α-1 antichymotrypsin. Note that both protein (α-1 antichymotrypsin) and chemical (chymostatin) molecules were efficient to decrease degradation of fibronectin.

### Example VI

### Inhibition of Cathepsin D-Like Protease Activity by a Specific Aspartate-Type Protease Inhibitor in Potato Leaf Extract

Materials and Methods

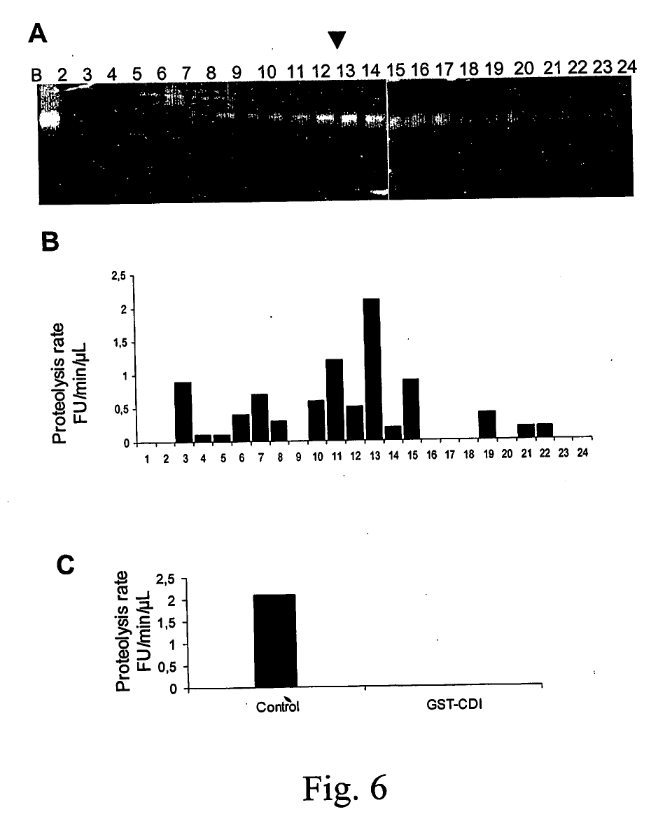

Similarly to Example V, soluble proteins were prepared from potato (cultivar Kennebec) leaves, separated by Mono-Q chromatography, and submitted to gelatin/PAGE (FIG. 6A), as described in FIG. 5A. Protease activity was determined for each chromatographic fraction by fluorimetry using a cathepsin D-specific substrate (MOCAc-Gly-Lys-Pro-Ile-Leu-Phe-Phe-Arg-Leu-Lys(Dnp)-D-Arg-NH2) at a final concentration of 6 μM (FIG. 6B). As depicted in FIG. 6C, protease activity in the potato leaf protein fraction showing the highest cathepsin D-like activity (fraction # 13) was dramatically altered by the aspartate-type inhibitor tomato cathepsin D inhibitor ‘CDI’, identifying CDI-sensitive proteases as interesting targets for the development of strategies aimed at protecting protein integrity via the inhibition of the plant's endogenous proteases. Noteworthy, our data also show that the inhibition of a single protease (or protease group) may be sufficient to protect a significant part of the proteins present in crude extracts, despite the presence of other (insensitive) proteases in the medium.

### Example VII

### Stabilization of Recombinant Proteins by the Ectopic Expression of a Tomato Cathepsin D Inhibitor in Potato

Materials and Methods

To assess the impact of ectopically expressing a recombinant protease inhibitor in the plant on the activity of endogenous proteases during extraction (ex vitro), a cathepsin D inhibitor from tomato, tomato CDI (Werner et al. 1993, Plant Physiology 103:1473), was integrated into an expression vector and stably expressed into potato (cultivar Kennebec). Transgenic controls expressing the selection marker neomycine phosphotransferase (NPTII) but no CDI were also devised by integrating the CDI transgene with no promoter. The tomato CDI-encoding DNA sequence was isolated from the expression vector pGEX-3X/CDI (Brunelle et al. 1999, Arch. Insect Biochem. Physiol. 42:88-98) by digestion with BamHI and EcoRI, and subcloned between the BamHI and EcoRI cloning sites of the commercial vector pCambia 2300 (CAMBIA, Canberra, Australia). The CaMV 35S promoter was isolated from the commercial plasmid pBI-121 (Clontech, Palo Alto, Calif.) using a BamHI/SalI treatment, and then ligated between the BamHI and SalI cloning sites of the pCambia construct including the CDI transgene. Transgenic controls (SPCD lines) expressing the selection marker neomycine phosphotransferase (NPTII) but no CDI were devised by integrating the CDI transgene with no promoter. Transformation of potato plants were performed as indicated in Example I. Expression of the CDI transgene in transgenic lines was monitored by RT-PCR and Northern blotting, using total RNA extracted from the fourth, fifth and sixth leaves of nptII transgene-positive plants, as described by Logemann et al. (1987, Anal Biochem. 163:16-20).

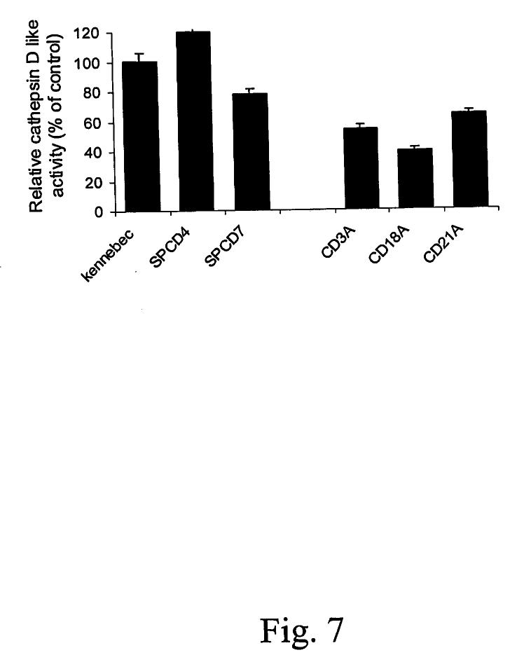

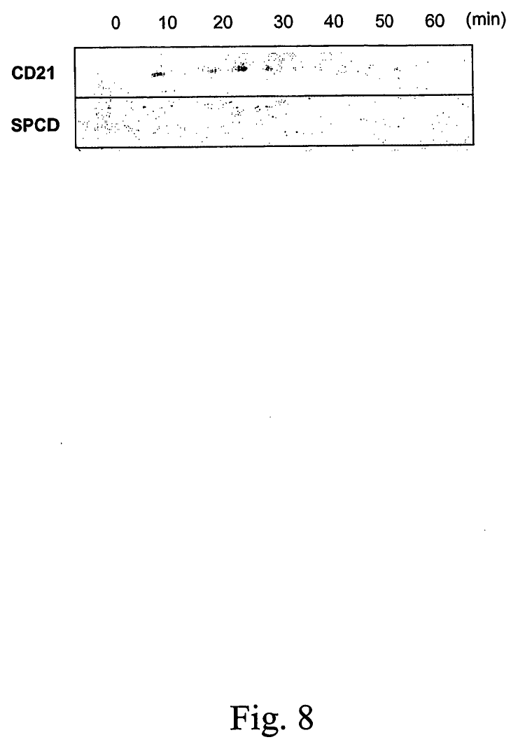

The cathepsin D-like activity was determined in transgenic potato plant expressing low (Kennebec, SPCD4 and SPCD7) or high (CD3A, CD18A, CD21A) levels of CDI mRNA. Leaf proteins were extracted as in Example IV. Fluorimetric assays of cathepsin-D activity were performed as in Example VI. As shown in FIG. 7, cathepsin D-like activity was significantly lowered in transgenic potato line expressing the CDI transgene. As shown by Western blotting with an appropriate polyclonal antibody (FIG. 8), degradation of the recombinant marker protein NPTII by potato leaf proteases in crude protein extracts from transgenic lines expressing high levels of recombinant CDI mRNA (clone 21A) was significantly decreased, compared to the degradation pattern observed for the transgenic control line, SPCD4. While NPTII protein can still be detected in the CD21 transgenic plant extract expressing the CDI at high level after 50 min, it is totally degraded only 10 min after incubation in the control line containing the promoter less CDI construct (SPCD). From a practical viewpoint, this observation shows that aspartate proteinase activity in potato leaf extracts is effectively inhibited by tomato CDI, protecting the recombinant protein from hydrolysis by this enzyme.

As described above for alfalfa and potato, plant leaf cells contain a considerable amount of non-specific proteases released in the medium during extraction. It is generally assumed that most of non-specific proteolytic activities in plant leaf cells are accounted for by proteases active in the acidic-to-mildly acidic pH range, usually belonging to the cysteine and aspartate class of proteolytic enzymes. It appears from the data presented here that different types of proteases—for instance CDI and chymostatin-sensitive proteases—may have a significant impact on the stability of useful proteins. As most non-specific proteases are often found in cell compartments other than the cytoplasm, inhibitors active against these proteases (e.g. tomato CDI or α1-antichymotrypsin) may be expressed in the cytoplasmic compartment of leaf cells (or elsewhere) in such a way that they do not negatively interfere with the host plant's metabolism in vivo, then ready to act against endogenous proteases after cell breakage during the recovery process.

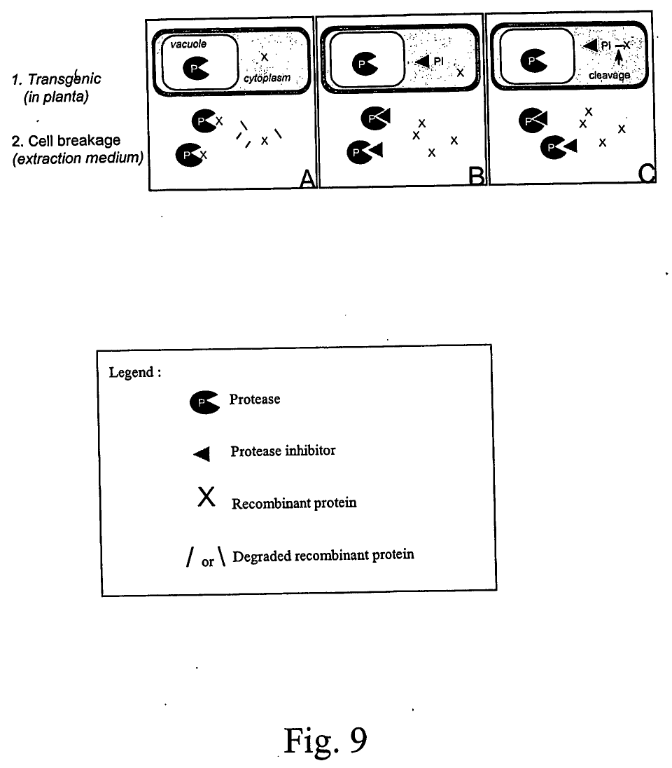

In practice, two different strategies may be used to achieve this goal (FIGS. 9B and 9C). A first strategy consists in developing transgenic lines of alfalfa expressing an appropriate protease inhibitor, and then using this line as an “anti-proteolysis” (or “low-proteolysis”) factory for the generation of double transformants expressing useful proteins (FIG. 9B). A second strategy consists in designing fusion proteins comprising the candidate protease inhibitor and the protein of interest, linked by a protease-sensitive cleavage site allowing cleavage of the fusion and recovery of the free proteins (FIG. 9C). For Strategy 1, the protease inhibitor-expressing transgenic line then serves as a ‘universal’ factory for the production of heterologous proteins in alfalfa. Strategy 2 is more specific, as gene fusions are devised for each particular protein to express, but a single transformation step is sufficient to protect the protein. In both cases the companion inhibitor is present in the plant's cells in vivo, then ready to inhibit any active plant target protease after disruption of cell compartments during extraction.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for increasing the recovery yield of a recombinant protein in plant cells without significantly altering the natural physiology of said plant cells, comprising neutralizing the activity or the action of at least one plant protease involved in the degradation of said recombinant protein with an inhibitor released from said plant cell at the time said plant cells are disrupted.

2. The method of claim 1, wherein said plant cells are from a plant or from an in vitro culture.

3. The method of claim 1 wherein said neutralizing is partial or total.

4. The method of claim 1 wherein said neutralizing occurs when processing said plant cells for extracting said recombinant protein.

5. The method of claim 1, wherein said plant cells are disrupted when performing a process for extracting said recombinant protein.

6. The method of claim 1, wherein said protease is selected from the group consisting of a cysteine protease, an aspartate, protease, a metallo protease, a serine protease, a threonine protease, and a multispecific protease.

7. The method of claim 1, wherein said inhibitor is recombinantly produced in said plant cells transformed with an expression cassette comprising a promoter operably linked thereto.

8. The method of claim 1, wherein said inhibitor is linked to a leader peptide, a signal peptide or an anchorage peptide or a protein to lead or anchor said inhibitor to a cell part or extracellular compartment in a manner to protect said recombinant protein from the activity of a plant protease during the extraction process.

9. The method of claim 7, wherein said inhibitor does not interfere with the activity of said protease to preserve the physiology or the growth of said plant cells or plant containing said plant cells.

10. The method of claim 7, wherein said cell part is an organelle selected from the group consisting of a mitochondria, a chloroplast, a storage vacuole, the endoplasmic reticulum, and the cytosol.

11. The method of claim 7, wherein said inhibitor is selected from the group consisting of an, antibody or a fragment thereof, a sens-mRNA or anti-sens mRNA, an inhibitor of transcription or a regulator thereof, an inhibitor of translation or a regulator thereof, an inhibitor of leading or signal peptide, an inhibitor of metabolic acquisition of activity of a protease, a protease-specific protease, and an affinity peptide protease leading to segregation to said protease into an organelle or a cell compartment.

12. The method of claim 8, wherein said genetically altered plant is an alfalfa or a potatoe.

13. The method of claim 1, wherein said protease is chymostatin-sensitive serine protease.

14. The method of claim 1, wherein said protease is a cystatin-sensitive cysteine protease.

15. The method of claim 1, wherein said inhibitor is a protease inhibitor.

16. The method of claim 1, wherein said plant cells are genetically altered

17. The method of claim 1, wherein said neutralizing is performed by an inhibitor encoded by a gene under control of a constitutive or an inducible promoter or a tissue or development specific promoter.

18. The method of claim 3 or claim 5, wherein said recombinant protein or inhibitor are produced in nucleus or plastids of said plant cells.

19. A method for increasing the recovery yield of a recombinant protein in a plant comprising the steps of:
a) allowing production of a recombinant protein in plant cells genetically altered for modulating at least one genetic or metabolic reaction to partially or totally neutralize action or activity of at least one protease at the time of disrupting of said plant cells; and
b) recovering said recombinant protein after disrupting of said plant cells.

20. The method of claim 19, wherein said plant cells are from a plant or from in vitro culture.

21. The method of claim 19, wherein said action or activity of said protease is neutralized by inhibiting its transcription or translation into an active protease, or by an inhibitor produced by said plant cells, or linking said recombinant protein with a peptide or protein in manner to protect said recombinant protein from the action or activity of said protease.

22. A plant cell or a plant genetically altered to modulate at least one genetic or metabolic reaction to partially or totally neutralize the action or activity of at least one protease for improving the recovery of a recombinant protein from said plant cell or plant at the time said plant cell or cells of said plant are disrupted.

23. The plant cell or plant of claim 22, wherein said modulation inhibits the transcription or translation of a gene encoding for a protease, or neutralizes a protease with a protease inhibitor produced in said plant or plant cell.

24. The plant cell or plant of claim 22, wherein said recombinant protein or protease inhibitor is linked to a leader peptide, a signal peptide or protein in manner to improve protection of said recombinant protein from at least one protease.
